(19) United States
(12) Patent Application Publication
(10) Pub. No.: US 2025/0049442 A1
(43) Pub. Date: Feb. 13, 2025

(54) ROTARY MOTION PASSIVE END EFFECTOR FOR SURGICAL ROBOTS IN ORTHOPEDIC SURGERIES

(71) Applicant: GLOBUS MEDICAL, INC., AUDUBON, PA (US)

(72) Inventor: Timothy Blackwell, Ft. Pierce, FL (US)

(21) Appl. No.: 18/922,959

(22) Filed: Oct. 22, 2024

Related U.S. Application Data
(63) Continuation of application No. 18/499,410, filed on Nov. 1, 2023, now Pat. No. 12,121,240.
Application No. 18/499,410 is a continuation of application No. 18/056,979, filed on Nov. 18, 2022, now Pat. No. 11,844,532.
Application No. 18/056,979 is a continuation of application No. 16/601,096, filed on Oct. 14, 2019, now Pat. No. 11,510,684.

(51) Int. Cl. A61B 17/14; A61B 17/00; A61B 34/00; A61B 34/30; A61B 90/50

(52) U.S. Cl. CPC A61B 17/147; A61B 34/25; A61B 34/30; A61B 90/50; A61B 2017/00057; A61B 2017/00075; A61B 2017/00398; A61B 2017/00477; A61B 2017/00907; A61B 2034/252; A61B 2034/254; A61B 2090/502

(57) ABSTRACT

A passive end effector of a surgical system includes a base connected to a rotational disk, and a saw attachment connected to the rotational disk. The base is attached to an end effector coupler of a robot arm positioned by a surgical robot, and includes a base arm extending away from the end effector coupler. The rotational disk is rotatably connected to the base arm and rotates about a first location on the rotational disk relative to the base arm. The saw attachment is rotatably connected to the rotational disk and rotates about a second location on the rotational disk. The first location on the rotational disk is spaced apart from the second location on the rotational disk. The saw attachment is configured to connect to a surgical saw including a saw blade configured to oscillate for cutting. The saw attachment rotates about the rotational disk and the rotational disk rotates about the base arm to constrain cutting of the saw blade to a range of movement along arcuate paths within a cutting plane.

### CROSS-REFERENCE TO RELATED APPLICATIONS

The present application is a continuation of U.S. patent application Ser. No. 18/499,410, filed on Nov. 1, 2023, which is a continuation of U.S. patent application Ser. No. 18/056,979, filed on Nov. 18, 2022 (published as U.S. Pat. Pub. No. 2023-0077678), which is a continuation of U.S. patent application Ser. No. 16/601,096, filed Oct. 14, 2019, now U.S. Pat. No. 11,510,684, all of which are incorporated herein by reference.

### FIELD

The present disclosure relates to medical devices and systems, and more particularly, robotic systems and related end effectors for controlling cutting of anatomical structures of a patient, and related methods and devices.

### BACKGROUND

There are a number of surgical interventions requiring osteotomy, i.e. cutting an anatomical structure such as a bone along a target plane. Total knee arthroplasty typically requires cutting both the femoral epiphysis and tibial epiphysis in order to remove the damaged bone and cartilage and install a knee prosthesis. A surgeon may perform five or more cuts on the femur and one or more cuts on the tibia using an oscillating surgical saw.

During orthopedic surgeries, including joints and knees, it is important to accurately align and stabilize the saw while cutting a desired location on a bone. The surgeon's limited visibility to the surgical site combined with the difficultly in controlling movement of the saw creates a risk that an undesired part of a bone or adjacent tissue becomes cut. Vibrations generated by the saw while cutting can reduce the accuracy of the cuts. During knee surgery, the precision of a bone cut (planar cuts) affects how precisely the implant can be connected to the exposed bone.

During some knee surgeries, a jig is screwed to a bone for guiding a surgeon's movement of a saw while cutting. Error in jig placement and limited stability of the saw blade during cutting can limit precision of the cuts. Moreover, contact between the saw blade and the jig can generate debris which risks entering the patient.

### SUMMARY

Some embodiments of the present disclosure are directed to a passive end effector of a surgical system that includes a base connected to a rotational disk and further includes a saw attachment connected to the rotational disk. The base is attached to an end effector coupler of a robot arm positioned by a surgical robot, and includes a base arm extending away from the end effector coupler. The rotational disk is rotatably connected to the base arm and rotates about a first location on the rotational disk relative to the base arm. The saw attachment is rotatably connected to the rotational disk and rotates about a second location on the rotational disk. The first location on the rotational disk is spaced apart from the second location on the rotational disk. The saw attachment is configured to connect to a surgical saw including a saw blade configured to oscillate for cutting. The saw attachment rotates about the rotational disk and the rotational disk rotates about the base arm to constrain cutting of the saw blade to a range of movement along arcuate paths within a cutting plane.

Some other embodiments of the present disclosure are directed to a surgical system that includes a tracking system, a surgical robot, and a passive end effector. The tracking system is configured to determine a pose of an anatomical structure to be cut by a saw blade and to determine a range of movement of the saw blade along arcuate paths within a cutting plane. The surgical robot includes a robot base, a robot arm rotatably connected to the robot base, at least one motor operatively connected to move the robot arm relative to the robot base, and at least one controller connected to the at least one motor. The passive end effector includes a base, a rotational disk, and a saw attachment. The base is configured to attach to an end effector coupler of the robot arm and includes a base arm extending away from the end effector coupler. The rotational disk is rotatably connected to the base arm and rotates about a first location on the rotational disk relative to the base arm. The saw attachment is rotatably connected to the rotational disk and rotates about a second location on the rotational disk. The first location on the rotational disk is spaced apart from the second location on the rotational disk. The saw attachment is configured to connect to a surgical saw including a saw blade configured to oscillate for cutting. The saw attachment rotates about the rotational disk and the rotational disk rotates about the base arm to constrain cutting of the saw blade to a range of movement along arcuate paths within a cutting plane.

The at least one controller is configured to determine a pose of a target plane based on a surgical plan defining where the anatomical structure is to be cut and based on the pose of the anatomical structure. The at least one controller is further configured to generate steering information based on comparison of the pose of the target plane and the determined range of movement of the saw blade along arcuate paths within the cutting plane. The steering information indicates where the passive end effector needs to be moved to position the cutting plane of the saw blade to be aligned with the target plane and so the saw blade is within the range of movement from the anatomical structure to be cut.

Other surgical systems, passive end effectors, and corresponding methods and computer program products according to embodiments will be or become apparent to one with skill in the art upon review of the following drawings and detailed description. It is intended that all such surgical systems, passive end effectors, and corresponding methods and computer program products be included within this description, be within the scope of the present disclosure, and be protected by the accompanying claims. Moreover, it is intended that all embodiments disclosed herein can be implemented separately or combined in any way and/or combination.

### BRIEF DESCRIPTION OF THE DRAWINGS

The accompanying drawings, which are included to provide a further understanding of the disclosure and are incorporated in a constitute a part of this application, illustrate certain non-limiting embodiments of inventive concepts. In the drawings:

FIG. 1 illustrates an embodiment of a surgical system according to some embodiments of the present disclosure;

FIG. 2 illustrates a surgical robot component of the surgical system of FIG. 1 according to some embodiments of the present disclosure;

FIG. 3 illustrates a camera tracking system component of the surgical system of FIG. 1 according to some embodiments of the present disclosure;

FIG. 4 illustrates an embodiment of a passive end effector that is connectable to a robot arm and configured according to some embodiments of the present disclosure;

FIG. 5 illustrates a medical operation in which a surgical robot and a camera system are disposed around a patient;

FIG. 6 illustrates an embodiment of an end effector coupler of a robot arm configured for connection to a passive end effector according to some embodiments of the present disclosure;

FIG. 7 illustrates an embodiment of a cut away of the end effector coupler of FIG. 6;

FIG. 8 illustrates a block diagram of components of a surgical system according to some embodiments of the present disclosure;

FIG. 9 illustrates a block diagram of a surgical system computer platform that includes a surgical planning computer which may be separate from and operationally connected to a surgical robot or at least partially incorporated therein according to some embodiments of the present disclosure;

FIG. 10 illustrates an embodiment of a C-Arm imaging device that can be used in combination with the surgical robot and passive end effector in accordance with some embodiments of the present disclosure;

FIG. 11 illustrates an embodiment of an O-Arm imaging device that can be used in combination with the surgical robot and passive end effector in accordance with some embodiments of the present disclosure; and

FIG. 12 illustrates an exploded view of components of a passive end effector that can be connected to a surgical saw and which are configured in accordance with some embodiments of the present disclosure;

FIG. 13 illustrates the assembled passive end effector of FIG. 12 connected to a surgical saw and configured in accordance with some embodiments of the present disclosure;

FIGS. 14a-14d illustrate a sequence of top views of the passive end effector and surgical saw of FIG. 13 in which the surgical saw is rotated about the rotational disk and the rotational disk is rotated about the base arm to provide a range of movement of the saw blade along arcuate paths within a cutting plane in accordance with some embodiments of the present disclosure;

FIG. 15 illustrates a combination of the top views of the passive end effector and surgical saw of FIGS. 14a and 14c to show a range of the movement of the saw blade along a horizontal axis that is provided by rotation of the surgical saw about the rotational disk and rotation of the rotational disk about the base arm;

FIG. 16 illustrates a light source, a tracking ring, and a light pulse detector configured in accordance with one embodiment to provide input to a tracking system for determining an arcuate path through which the saw blade moves; and

FIG. 17 illustrates a light source, a tracking ring, and a light pulse detector configured in accordance with another embodiment to provide input to a tracking system for determining an arcuate path through which the saw blade moves.

### DETAILED DESCRIPTION

Inventive concepts will now be described more fully hereinafter with reference to the accompanying drawings, in which examples of embodiments of inventive concepts are shown. Inventive concepts may, however, be embodied in many different forms and should not be construed as limited to the embodiments set forth herein. Rather, these embodiments are provided so that this disclosure will be thorough and complete, and will fully convey the scope of various present inventive concepts to those skilled in the art. It should also be noted that these embodiments are not mutually exclusive. Components from one embodiment may be tacitly assumed to be present or used in another embodiment.

Various embodiments disclosed herein are directed to improvements in operation of a surgical system when performing surgical interventions requiring osteotomy. A passive end effector is disclosed that is connectable to a robot arm positioned by a surgical robot. The passive end effector includes a base, a rotational disk, and a saw attachment. The base is configured to attach to an end effector coupler of the robot arm, and includes a base arm extending away from the end effector coupler. The rotational disk is rotatably connected to the base arm and rotates about a first location on the rotational disk relative to the base arm. The saw attachment is rotatably connected to the rotational disk and rotates about a second location on the rotational disk. The first location on the rotational disk is spaced apart from the second location on the rotational disk. The saw attachment is configured to connect to a surgical saw having a saw blade which is configured to oscillate for cutting. The saw attachment rotates about the rotational disk and the rotational disk rotates about the base arm to constrain cutting of the saw blade to a range of movement along arcuate paths within a cutting plane.

As will be further explained below, the surgical robot can determine a pose of a target plane based on a surgical plan defining where an anatomical structure is to be cut and based on a pose of the anatomical structure. The surgical robot can also generate steering information based on comparison of the pose of the target plane and a determined range of movement of the saw blade along arcuate paths within the cutting plane. The steering information indicates where the passive end effector needs to be moved to position the cutting plane of the saw blade to be aligned with the target plane and so the saw blade is within the range of movement from the anatomical structure to be cut. The steering information can be used to automatically position the passive end effector relative to the anatomical structure and/or to provide guidance to an operator who positions the passive end effector relative to the anatomical structure.

These and other related embodiments can operate to improve the precision of the guidance of the saw blade compared to other robotic and manual (e.g., jigs) solutions for surgeries. The planar mechanisms of the passive end effector can allow the surgeon to concentrate on interpreting the direct force feedback while cutting bones using a surgical saw that is guided by the passive end effector, while the planar mechanisms constrain the cutting plane to be aligned with the target plane. The surgeon may also more accurately monitor and control the speed of bone removal based on audio and/or visual notification feedback provided through the surgical robot.

These embodiments can provide guidance during joint surgeries and especially knee surgery with high precision, high rigidity, sufficient workspace and direct force feedback. As will be explained in detail below, a tracking system can be used to precisely align the cutting plane with the target plane for cutting a bone. High precision cuts may be achieved by the planar mechanisms constraining the cutting plane to remaining aligned with the target plane while a surgeon moves the saw blade along the cutting plane and directly senses force feedback of the saw blade cutting bone. Moreover, these embodiments can be rapidly deployed into surgical practices through defined changes in existing accepted surgery workflows.

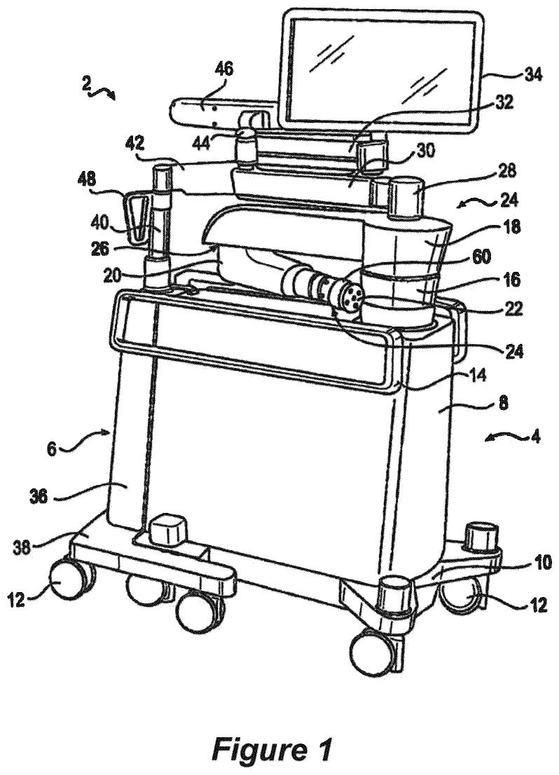

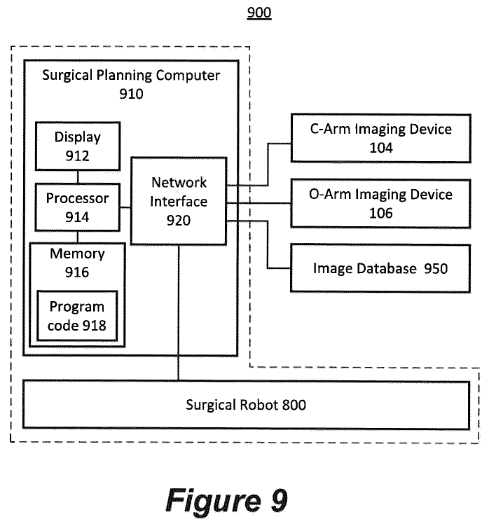

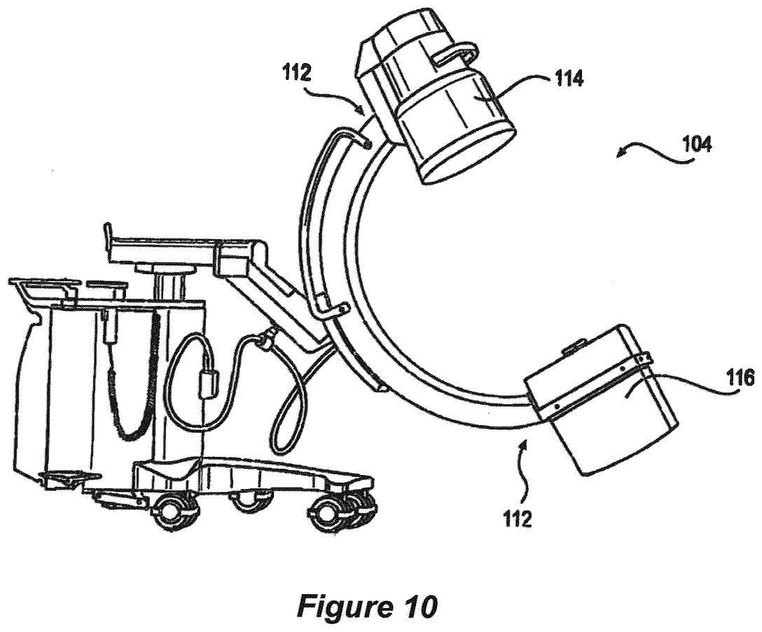

FIG. 1 illustrates an embodiment of a surgical system 2 according to some embodiments of the present disclosure. Prior to performance of an orthopedic surgical procedure, a three-dimensional (“3D”) image scan may be taken of a planned surgical area of a patient using, e.g., the C-Arm imaging device 104 of FIG. 10 or O-Arm imaging device 106 of FIG. 11, or from another medical imaging device such as a computed tomography (CT) image or MRI. This scan can be taken pre-operatively (e.g. few weeks before procedure, most common) or intra-operatively. However, any known 3D or 2D image scan may be used in accordance with various embodiments of the surgical system 2. The image scan is sent to a computer platform in communication with the surgical system 2, such as the surgical system computer platform 900 of FIG. 9 which includes the surgical robot 800 (e.g., robot 2 in FIG. 1) and a surgical planning computer 910. A surgeon reviewing the image scan(s) on a display device of the surgical planning computer 910 (FIG. 9) generates a surgical plan defining a target plane where an anatomical structure of the patient is to be cut. This plane is a function of patient anatomy constraints, selected implant and its size. In some embodiments, the surgical plan defining the target plane is planned on the 3D image scan displayed on a display device.

The surgical system 2 of FIG. 1 can assist surgeons during medical procedures by, for example, holding tools, aligning tools, using tools, guiding tools, and/or positioning tools for use. In some embodiments, surgical system 2 includes a surgical robot 4 and a camera tracking system 6. Both systems may be mechanically coupled together by any various mechanisms. Suitable mechanisms can include, but are not limited to, mechanical latches, ties, clamps, or buttresses, or magnetic or magnetized surfaces. The ability to mechanically couple surgical robot 4 and camera tracking system 6 can allow for surgical system 2 to maneuver and move as a single unit, and allow surgical system 2 to have a small footprint in an area, allow easier movement through narrow passages and around turns, and allow storage within a smaller area.

An orthopedic surgical procedure may begin with the surgical system 2 moving from medical storage to a medical procedure room. The surgical system 2 may be maneuvered through doorways, halls, and elevators to reach a medical procedure room. Within the room, the surgical system 2 may be physically separated into two separate and distinct systems, the surgical robot 4 and the camera tracking system 6. Surgical robot 4 may be positioned adjacent the patient at any suitable location to properly assist medical personnel. Camera tracking system 6 may be positioned at the base of the patient, at the patient shoulders, or any other location suitable to track the present pose and movement of the pose of tracks portions of the surgical robot 4 and the patient. Surgical robot 4 and camera tracking system 6 may be powered by an onboard power source and/or plugged into an external wall outlet.

Surgical robot 4 may be used to assist a surgeon by holding and/or using tools during a medical procedure. To properly utilize and hold tools, surgical robot 4 may rely on a plurality of motors, computers, and/or actuators to function properly. Illustrated in FIG. 1, robot body 8 may act as the structure in which the plurality of motors, computers, and/or actuators may be secured within surgical robot 4. Robot body 8 may also provide support for robot telescoping support arm 16. In some embodiments, robot body 8 may be made of any suitable material. Suitable material may be, but is not limited to, metal such as titanium, aluminum, or stainless steel, carbon fiber, fiberglass, or heavy-duty plastic. The size of robot body 8 may provide a solid platform supporting attached components, and may house, conceal, and protect the plurality of motors, computers, and/or actuators that may operate attached components.

Robot base 10 may act as a lower support for surgical robot 4. In some embodiments, robot base 10 may support robot body 8 and may attach robot body 8 to a plurality of powered wheels 12. This attachment to wheels may allow robot body 8 to move in space efficiently. Robot base 10 may run the length and width of robot body 8. Robot base 10 may be about two inches to about 10 inches tall. Robot base 10 may be made of any suitable material. Suitable material may be, but is not limited to, metal such as titanium, aluminum, or stainless steel, carbon fiber, fiberglass, or heavy-duty plastic or resin. Robot base 10 may cover, protect, and support powered wheels 12.

In some embodiments, as illustrated in FIG. 1, at least one powered wheel 12 may be attached to robot base 10. Powered wheels 12 may attach to robot base 10 at any location. Each individual powered wheel 12 may rotate about a vertical axis in any direction. A motor may be disposed above, within, or adjacent to powered wheel 12. This motor may allow for surgical system 2 to maneuver into any location and stabilize and/or level surgical system 2. A rod, located within or adjacent to powered wheel 12, may be pressed into a surface by the motor. The rod, not pictured, may be made of any suitable metal to lift surgical system 2. Suitable metal may be, but is not limited to, stainless steel, aluminum, or titanium. Additionally, the rod may comprise at the contact-surface-side end a buffer, not pictured, which may prevent the rod from slipping and/or create a suitable contact surface. The material may be any suitable material to act as a buffer. Suitable material may be, but is not limited to, a plastic, neoprene, rubber, or textured metal. The rod may lift powered wheel 10, which may lift surgical system 2, to any height required to level or otherwise fix the orientation of the surgical system 2 in relation to a patient. The weight of surgical system 2, supported through small contact areas by the rod on each wheel, prevents surgical system 2 from moving during a medical procedure. This rigid positioning may prevent objects and/or people from moving surgical system 2 by accident.

Moving surgical system 2 may be facilitated using robot railing 14. Robot railing 14 provides a person with the ability to move surgical system 2 without grasping robot body 8. As illustrated in FIG. 1, robot railing 14 may run the length of robot body 8, shorter than robot body 8, and/or may run longer the length of robot body 8. Robot railing 14 may be made of any suitable material. Suitable material may be, but is not limited to, metal such as titanium, aluminum, or stainless steel, carbon fiber, fiberglass, or heavy-duty plastic. Robot railing 14 may further provide protection to robot body 8, preventing objects and or personnel from touching, hitting, or bumping into robot body 8.

Robot body 8 may provide support for a Selective Compliance Articulated Robot Arm, hereafter referred to as a “SCARA.” A SCARA 24 may be beneficial to use within the surgical system 2 due to the repeatability and compactness of the robotic arm. The compactness of a SCARA may provide additional space within a medical procedure, which may allow medical professionals to perform medical procedures free of excess clutter and confining areas. SCARA 24 may comprise robot telescoping support 16, robot support arm 18, and/or robot arm 20. Robot telescoping support 16 may be disposed along robot body 8. As illustrated in FIG. 1, robot telescoping support 16 may provide support for the SCARA 24 and display 34. In some embodiments, robot telescoping support 16 may extend and contract in a vertical direction. Robot telescoping support 16 may be made of any suitable material. Suitable material may be, but is not limited to, metal such as titanium or stainless steel, carbon fiber, fiberglass, or heavy-duty plastic. The body of robot telescoping support 16 may be any width and/or height in which to support the stress and weight placed upon it.

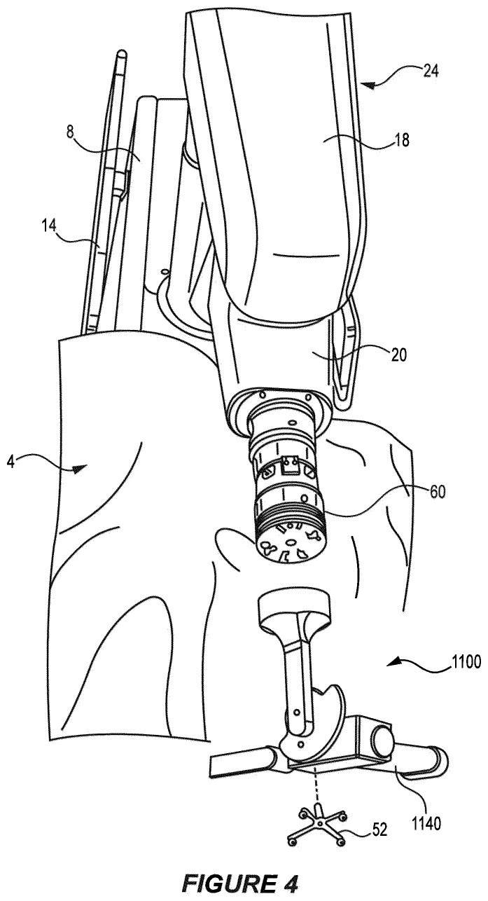

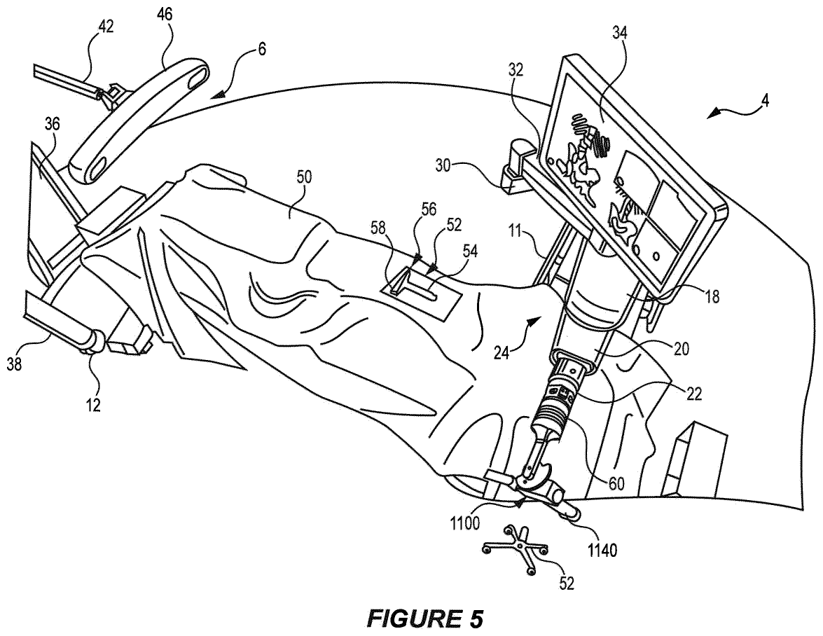

In some embodiments, medical personnel may move SCARA 24 through a command submitted by the medical personnel. The command may originate from input received on display 34 and/or a tablet. The command may come from the depression of a switch and/or the depression of a plurality of switches. Best illustrated in FIGS. 4 and 5, an activation assembly 60 may include a switch and/or a plurality of switches. The activation assembly 60 may be operable to transmit a move command to the SCARA 24 allowing an operator to manually manipulate the SCARA 24. When the switch, or plurality of switches, is depressed the medical personnel may have the ability to move SCARA 24 easily. Additionally, when the SCARA 24 is not receiving a command to move, the SCARA 24 may lock in place to prevent accidental movement by personnel and/or other objects. By locking in place, the SCARA 24 provides a solid platform upon which a passive end effector 1100 and connected surgical saw 1140, shown in FIGS. 4 and 4, are ready for use in a medical operation.

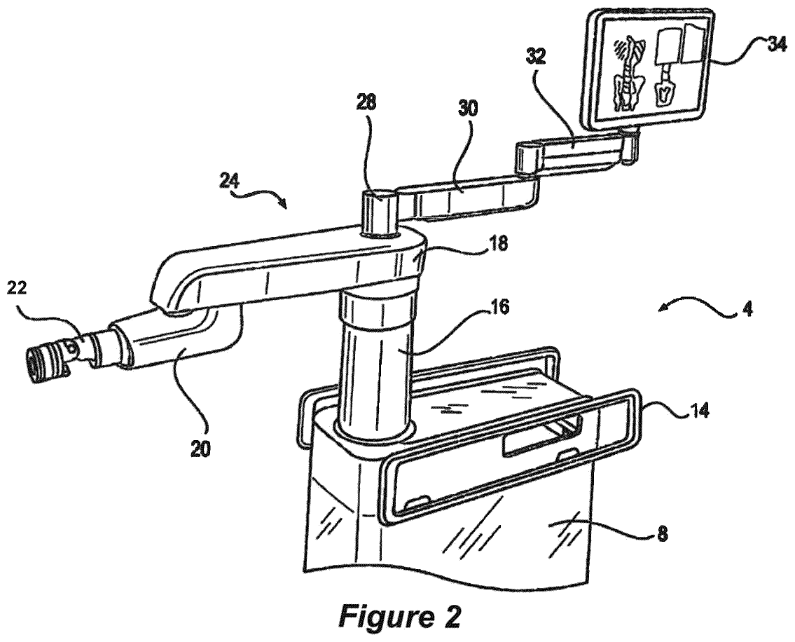

Robot support arm 18 may be disposed on robot telescoping support 16 by various mechanisms. In some embodiments, best seen in FIGS. 1 and 2, robot support arm 18 rotates in any direction in regard to robot telescoping support 16. Robot support arm 18 may rotate three hundred and sixty degrees around robot telescoping support 16. Robot arm 20 may connect to robot support arm 18 at any suitable location. Robot arm 20 may attach to robot support arm 16 by various mechanisms. Suitable mechanisms may be, but is not limited to, nuts and bolts, ball and socket fitting, press fitting, weld, adhesion, screws, rivets, clamps, latches, and/or any combination thereof. Robot arm 20 may rotate in any direction in regards to robot support arm 18, in embodiments, robot arm 20 may rotate three hundred and sixty degrees in regards to robot support arm 18. This free rotation may allow an operator to position robot arm 20 as planned.

The passive end effector 1100 in FIGS. 4 and 5 may attach to robot arm 20 in any suitable location. As will be explained in further detail below, the passive end effector 1100 includes a base, a first planar mechanism, and a second planar mechanism. The base is configured to attach to an end effector coupler 22 of the robot arm 20 positioned by the surgical robot 4. Various mechanisms by which the base can attach to the end effector coupler 22 can include, but are not limited to, latch, clamp, nuts and bolts, ball and socket fitting, press fitting, weld, adhesion, screws, rivets, and/or any combination thereof. The first planar mechanism extends between a rotatable connection to the base and a rotatable connection to a tool attachment mechanism. The second planar mechanism extends between a rotatable connection to the base and a rotatable connection to the tool attachment mechanism. The first and second planar mechanisms pivot about the rotatable connections to constrain movement of the tool attachment mechanism to a range of movement within a working plane. The tool attachment mechanism is configured to connect to a surgical saw 1140 having a saw blade. The surgical saw 1140 may be configured as a sagittal saw which oscillates the saw blade for cutting. The first and second planar mechanisms constrain a cutting plane of the saw blade to be parallel to the working plane.

The tool attachment mechanism may connect to the surgical saw 1140 through various mechanisms that can include, but are not limited to, a channel, a screw, nut and bolt, clamp, latch, tie, press fit, or magnet. In some embodiments, a dynamic reference array 52 is attached to the passive end effector 1100, e.g., to the tool attachment mechanism, and/or is attached to the surgical saw 1140. Dynamic reference arrays, also referred to as “DRAs” herein, are rigid bodies which may be disposed on a patient, the surgical robot, the passive end effector, and/or the surgical saw in a navigated surgical procedure. The camera tracking system 6 or other 3D localization system is configured to track in real-time the pose (e.g., positions and rotational orientations) of tracking markers of the DRA. The tracking markers include fiducials, such as the illustrated arrangement of balls. This tracking of 3D coordinates of tracking markers can allow the surgical system 2 to determine the pose of the DRA 52 in any space in relation to the target anatomical structure of the patient 50 in FIG. 5.

As illustrated in FIG. 1, a light indicator 28 may be positioned on top of the SCARA 24. Light indicator 28 may illuminate as any type of light to indicate “conditions” in which surgical system 2 is currently operating. For example, the illumination of green may indicate that all systems are normal. Illuminating red may indicate that surgical system 2 is not operating normally. A pulsating light may mean surgical system 2 is performing a function. Combinations of light and pulsation may create a nearly limitless amount of combinations in which to communicate the current operating conditions, states, or other operational indications. In some embodiments, the light may be produced by LED bulbs, which may form a ring around light indicator 28. Light indicator 28 may comprise a fully permeable material that may let light shine through the entirety of light indicator 28.

Light indicator 28 may be attached to lower display support 30. Lower display support 30, as illustrated in FIG. 2 may allow an operator to maneuver display 34 to any suitable location. Lower display support 30 may attach to light indicator 28 by any suitable mechanism. In embodiments, lower display support 30 may rotate about light indicator 28. In embodiments, lower display support 30 may attach rigidly to light indicator 28. Light indicator 28 may then rotate three hundred and sixty degrees about robot support arm 18. Lower display support 30 may be of any suitable length, a suitable length may be about eight inches to about thirty four inches. Lower display support 30 may act as a base for upper display support 32.

Upper display support 32 may attach to lower display support 30 by any suitable mechanism. Upper display support 32 may be of any suitable length, a suitable length may be about eight inches to about thirty four inches. In embodiments, as illustrated in FIG. 1, upper display support 32 may allow display 34 to rotate three hundred and sixty degrees in relation to upper display support 32. Likewise, upper display support 32 may rotate three hundred and sixty degrees in relation to lower display support 30.

Display 34 may be any device which may be supported by upper display support 32. In embodiments, as illustrated in FIG. 2, display 34 may produce color and/or black and white images. The width of display 34 may be about eight inches to about thirty inches wide. The height of display 34 may be about six inches to about twenty two inches tall. The depth of display 34 may be about one-half inch to about four inches.

In embodiments, a tablet may be used in conjunction with display 34 and/or without display 34. In embodiments, the table may be disposed on upper display support 32, in place of display 34, and may be removable from upper display support 32 during a medical operation. In addition the tablet may communicate with display 34. The tablet may be able to connect to surgical robot 4 by any suitable wireless and/or wired connection. In some embodiments, the tablet may be able to program and/or control surgical system 2 during a medical operation. When controlling surgical system 2 with the tablet, all input and output commands may be duplicated on display 34. The use of a tablet may allow an operator to manipulate surgical robot 4 without having to move around patient 50 and/or to surgical robot 4.

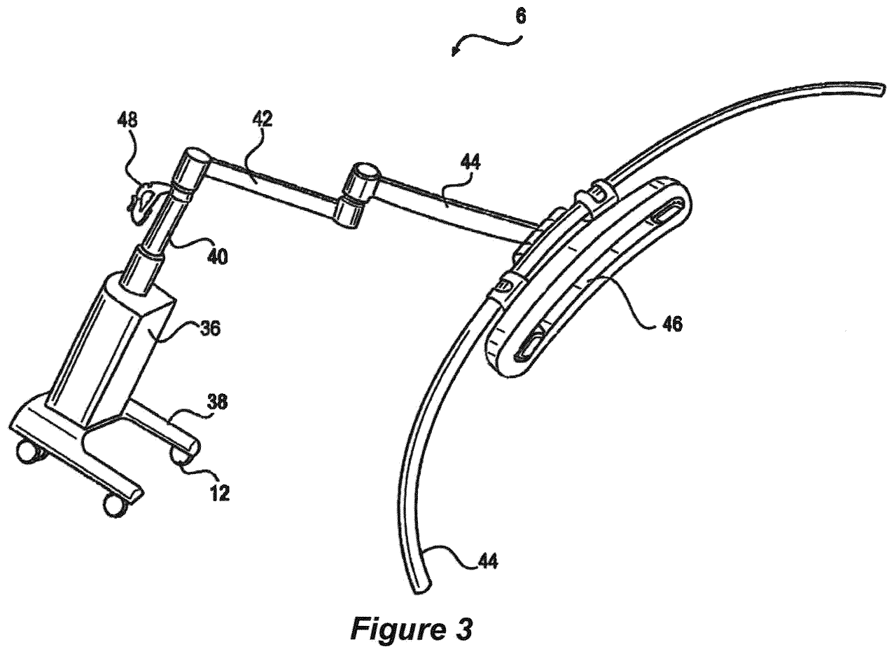

As illustrated in FIGS. 3 and 5, camera tracking system 6 works in conjunction with surgical robot 4 through wired or wireless communication networks. Referring to FIGS. 1, 3 and 5, camera tracking system 6 can include some similar components to the surgical robot 4. For example, camera body 36 may provide the functionality found in robot body 8. Robot body 8 may provide the structure upon which camera 46 is mounted. The structure within robot body 8 may also provide support for the electronics, communication devices, and power supplies used to operate camera tracking system 6. Camera body 36 may be made of the same material as robot body 8. Camera tracking system 6 may communicate directly to the tablet and/or display 34 by a wireless and/or wired network to enable the tablet and/or display 34 to control the functions of camera tracking system 6.

Camera body 36 is supported by camera base 38. Camera base 38 may function as robot base 10. In the embodiment of FIG. 1, camera base 38 may be wider than robot base 10. The width of camera base 38 may allow for camera tracking system 6 to connect with surgical robot 4. As illustrated in FIG. 1, the width of camera base 38 may be large enough to fit outside robot base 10. When camera tracking system 6 and surgical robot 4 are connected, the additional width of camera base 38 may allow surgical system 2 additional maneuverability and support for surgical system 2.

As with robot base 10, a plurality of powered wheels 12 may attach to camera base 38. Powered wheel 12 may allow camera tracking system 6 to stabilize and level or set fixed orientation in regards to patient 50, similar to the operation of robot base 10 and powered wheels 12. This stabilization may prevent camera tracking system 6 from moving during a medical procedure and may keep camera 46 from losing track of one or more DRAs 52 connected to an anatomical structure 54 and/or tool 58 within a designated area 56 as shown in FIGS. 3 and 5. This stability and maintenance of tracking enhances the ability of surgical robot 4 to operate effectively with camera tracking system 6. Additionally, the wide camera base 38 may provide additional support to camera tracking system 6. Specifically, a wide camera base 38 may prevent camera tracking system 6 from tipping over when camera 46 is disposed over a patient, as illustrated in FIGS. 3 and 5. Without the wide camera base 38, the outstretched camera 46 may unbalance camera tracking system 6, which may result in camera tracking system 6 falling over.

Camera telescoping support 40 may support camera 46. In some embodiments, telescoping support 40 moves camera 46 higher or lower in the vertical direction. Telescoping support 40 may be made of any suitable material in which to support camera 46. Suitable material may be, but is not limited to, metal such as titanium, aluminum, or stainless steel, carbon fiber, fiberglass, or heavy-duty plastic. Camera handle 48 may be attached to camera telescoping support 40 at any suitable location. Cameral handle 48 may be any suitable handle configuration. A suitable configuration may be, but is not limited to, a bar, circular, triangular, square, and/or any combination thereof. As illustrated in FIG. 1, camera handle 48 may be triangular, allowing an operator to move camera tracking system 6 into a planned position before a medical operation. In some embodiments, camera handle 48 is used to lower and raise camera telescoping support 40. Camera handle 48 may perform the raising and lowering of camera telescoping support 40 through the depression of a button, switch, lever, and/or any combination thereof.

Lower camera support arm 42 may attach to camera telescoping support 40 at any suitable location, in embodiments, as illustrated in FIG. 1, lower camera support arm 42 may rotate three hundred and sixty degrees around telescoping support 40. This free rotation may allow an operator to position camera 46 in any suitable location. Lower camera support arm 42 may be made of any suitable material in which to support camera 46. Suitable material may be, but is not limited to, metal such as titanium, aluminum, or stainless steel, carbon fiber, fiberglass, or heavy-duty plastic. Cross-section of lower camera support arm 42 may be any suitable shape. Suitable cross-sectional shape may be, but is not limited to, circle, square, rectangle, hexagon, octagon, or i-beam. The cross-sectional length and width may be about one to ten inches. Length of the lower camera support arm may be about four inches to about thirty-six inches. Lower camera support arm 42 may connect to telescoping support 40 by any suitable mechanism. Suitable mechanism may be, but is not limited to, nuts and bolts, ball and socket fitting, press fitting, weld, adhesion, screws, rivets, clamps, latches, and/or any combination thereof. Lower camera support arm 42 may be used to provide support for camera 46. Camera 46 may be attached to lower camera support arm 42 by any suitable mechanism. Suitable mechanism may be, but is not limited to, nuts and bolts, ball and socket fitting, press fitting, weld, adhesion, screws, rivets, and/or any combination thereof. Camera 46 may pivot in any direction at the attachment area between camera 46 and lower camera support arm 42. In embodiments a curved rail 44 may be disposed on lower camera support arm 42.

Curved rail 44 may be disposed at any suitable location on lower camera support arm 42. As illustrated in FIG. 3, curved rail 44 may attach to lower camera support arm 42 by any suitable mechanism. Suitable mechanism may be, but are not limited to nuts and bolts, ball and socket fitting, press fitting, weld, adhesion, screws, rivets, clamps, latches, and/or any combination thereof. Curved rail 44 may be of any suitable shape, a suitable shape may be a crescent, circular, oval, elliptical, and/or any combination thereof. In embodiments, curved rail 44 may be any appropriate length. An appropriate length may be about one foot to about six feet. Camera 46 may be moveably disposed along curved rail 44. Camera 46 may attach to curved rail 44 by any suitable mechanism. Suitable mechanism may be, but are not limited to rollers, brackets, braces, motors, and/or any combination thereof. Motors and rollers, not illustrated, may be used to move camera 46 along curved rail 44. As illustrated in FIG. 3, during a medical procedure, if an object prevents camera 46 from viewing one or more DRAs 52, the motors may move camera 46 along curved rail 44 using rollers. This motorized movement may allow camera 46 to move to a new position that is no longer obstructed by the object without moving camera tracking system 6. While camera 46 is obstructed from viewing DRAs 52, camera tracking system 6 may send a stop signal to surgical robot 4, display 34, and/or a tablet. The stop signal may prevent SCARA 24 from moving until camera 46 has reacquired DRAs 52. This stoppage may prevent SCARA 24 and/or end effector coupler 22 from moving and/or using medical tools without being tracked by surgical system 2.

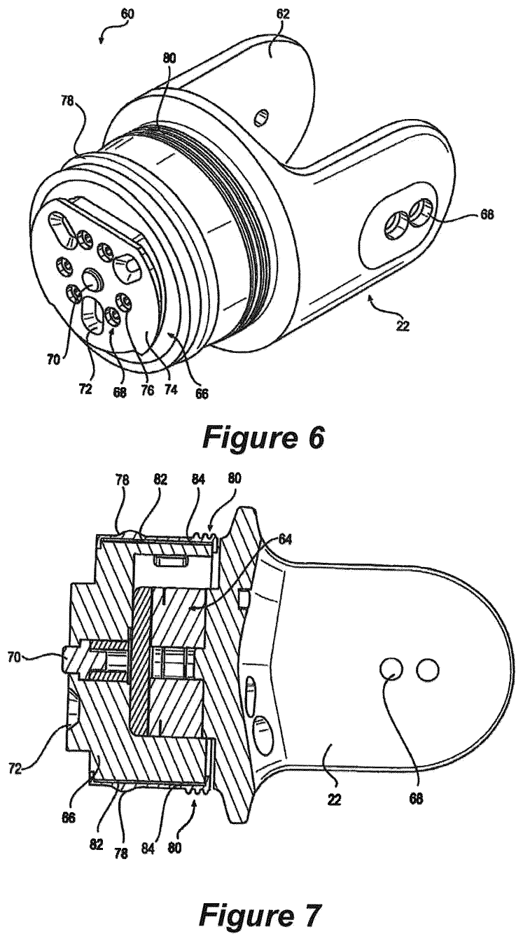

End effector coupler 22, as illustrated in FIG. 6, is configured to connect various types of passive end effectors to surgical robot 4 of FIG. 2. End effector coupler 22 can include a saddle joint 62, an activation assembly 60, a load cell 64 (FIG. 7), and a connector 66. Saddle joint 62 may attach end effector coupler 22 to SCARA 24. Saddle joint 62 may be made of any suitable material. Suitable material may be, but is not limited to metal such as titanium, aluminum, or stainless steel, carbon fiber, fiberglass, or heavy-duty plastic. Saddle joint 62 may be made of a single piece of metal which may provide end effector with additional strength and durability. The saddle joint 62 may attach to SCARA 24 by an attachment point 68. There may be a plurality of attachment points 68 disposed about saddle joint 62. Attachment points 68 may be sunk, flush, and/or disposed upon saddle joint 62. In some examples, screws, nuts and bolts, and/or any combination thereof may pass through attachment point 68 and secure saddle joint 62 to SCARA 24. The nuts and bolts may connect saddle joint 62 to a motor, not illustrated, within SCARA 24. The motor may move saddle joint 62 in any direction. The motor may further prevent saddle joint 62 from moving from accidental bumps and/or accidental touches by actively servoing at the current location or passively by applying spring actuated brakes.

The end effector coupler 22 can include a load cell 64 interposed between the saddle join 62 and a connected passive end effector. Load cell 64, as illustrated in FIG. 7 may attach to saddle joint 62 by any suitable mechanism. Suitable mechanism may be, but is not limited to, screws, nuts and bolts, threading, press fitting, and/or any combination thereof.

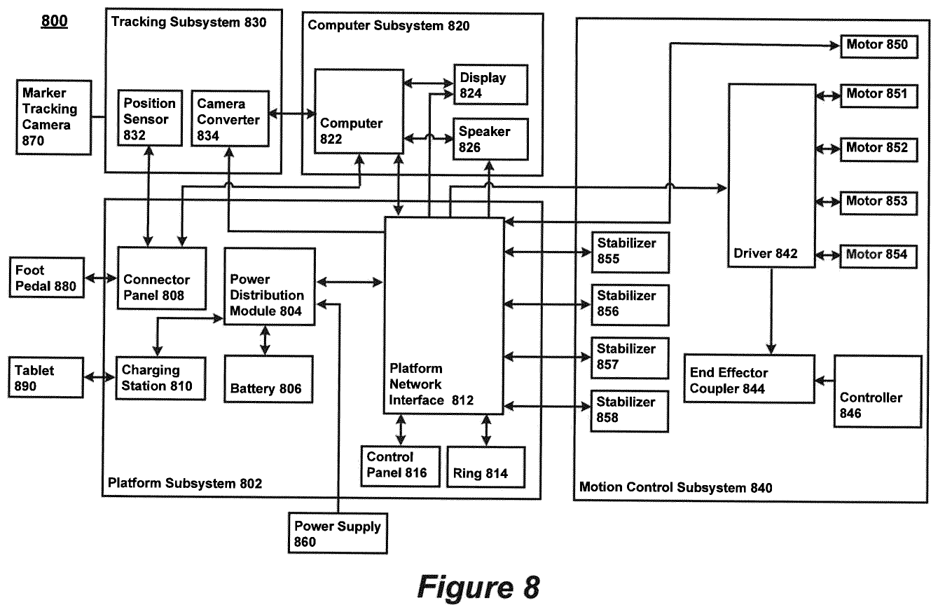

FIG. 8 illustrates a block diagram of components of a surgical system 800 according to some embodiments of the present disclosure. Referring to FIGS. 7 and 8, load cell 64 may be any suitable instrument used to detect and measure forces. In some examples, load cell 64 may be a six axis load cell, a three-axis load cell or a uniaxial load cell. Load cell 64 may be used to track the force applied to end effector coupler 22. In some embodiments the load cell 64 may communicate with a plurality of motors 850, 851, 852, 853, and/or 854. As load cell 64 senses force, information as to the amount of force applied may be distributed from a switch array and/or a plurality of switch arrays to a controller 846. Controller 846 may take the force information from load cell 64 and process it with a switch algorithm. The switch algorithm is used by the controller 846 to control a motor driver 842. The motor driver 842 controls operation of one or more of the motors. Motor driver 842 may direct a specific motor to produce, for example, an equal amount of force measured by load cell 64 through the motor. In some embodiments, the force produced may come from a plurality of motors, e.g., 850-854, as directed by controller 846. Additionally, motor driver 842 may receive input from controller 846. Controller 846 may receive information from load cell 64 as to the direction of force sensed by load cell 64. Controller 846 may process this information using a motion controller algorithm. The algorithm may be used to provide information to specific motor drivers 842. To replicate the direction of force, controller 846 may activate and/or deactivate certain motor drivers 842. Controller 846 may control one or more motors, e.g. one or more of 850-854, to induce motion of passive end effector 1100 in the direction of force sensed by load cell 64. This force-controlled motion may allow an operator to move SCARA 24 and passive end effector 1100 effortlessly and/or with very little resistance. Movement of passive end effector 1100 can be performed to position passive end effector 1100 in any suitable pose (i.e., location and angular orientation relative to defined three-dimensional (3D) orthogonal reference axes) for use by medical personnel.

Connector 66 is configured to be connectable to the base of the passive end effector 1100 and is connected to load cell 64. Connector 66 can include attachment points 68, a sensory button 70, tool guides 72, and/or tool connections 74. There may be a plurality of attachment points 68 as shown in FIG. 6. Attachment points 68 may connect connector 66 to load cell 64. Attachment points 68 may be sunk, flush, and/or disposed upon connector 66. Attachment points 68 and 76 can be used to attach connector 66 to load cell 64 and/or to passive end effector 1100. In some examples, Attachment points 68 and 76 may include screws, nuts and bolts, press fittings, magnetic attachments, and/or any combination thereof.

As illustrated in FIG. 6, a sensory button 70 may be disposed about center of connector 66. Sensory button 70 may be depressed when a passive end effector 1100 is connected to SCARA 24. Depression of sensory button 70 may alert surgical robot 4, and in turn medical personnel, that a passive end effector 1100 has been attached to SCARA 24. As illustrated in FIG. 6, guides 72 may be used to facilitate proper attachment of passive end effector 1100 to SCARA 24. Guides 72 may be sunk, flush, and/or disposed upon connector 66. In some examples there may be a plurality of guides 72 and may have any suitable patterns and may be oriented in any suitable direction. Guides 72 may be any suitable shape to facilitate attachment of passive end effector 1100 to SCARA 24. A suitable shape may be, but is not limited to, circular, oval, square, polyhedral, and/or any combination thereof. Additionally, guides 72 may be cut with a bevel, straight, and/or any combination thereof.

Connector 66 may have attachment points 74. As illustrated in FIG. 6, attachment points 74 may form a ledge and/or a plurality of ledges. Attachment points 74 may provide connector 66 a surface upon which passive end effector 1100 may clamp. In some embodiments, attachment points 74 are disposed about any surface of connector 66 and oriented in any suitable manner in relation to connector 66.

Activation assembly 60, best illustrated in FIGS. 6 and 7, may encircle connector 66. In some embodiments, activation assembly 60 may take the form of a bracelet that wraps around connector 66. In some embodiments, activation assembly 60, may be located in any suitable area within surgical system 2. In some examples, activation assembly 60 may be located on any part of SCARA 24, any part of end effector coupler 22, may be worn by medical personnel (and communicate wirelessly), and/or any combination thereof. Activation assembly 60 may be made of any suitable material. Suitable material may be, but is not limited to neoprene, plastic, rubber, gel, carbon fiber, fabric, and/or any combination thereof. Activation assembly 60 may comprise of a primary button 78 and a secondary button 80. Primary button 78 and secondary button 80 may encircle the entirety of connector 66.

Primary button 78 may be a single ridge, as illustrated in FIG. 6, which may encircle connector 66. In some examples, primary button 78 may be disposed upon activation assembly 60 along the end farthest away from saddle joint 62. Primary button 78 may be disposed upon primary activation switch 82, best illustrated on FIG. 7. Primary activation switch 82 may be disposed between connector 66 and activation assembly 60. In some examples, there may be a plurality of primary activation switches 82, which may be disposed adjacent and beneath primary button 78 along the entire length of primary button 78. Depressing primary button 78 upon primary activation switch 82 may allow an operator to move SCARA 24 and end effector coupler 22. As discussed above, once set in place, SCARA 24 and end effector coupler 22 may not move until an operator programs surgical robot 4 to move SCARA 24 and end effector coupler 22, or is moved using primary button 78 and primary activation switch 82. In some examples, it may require the depression of at least two non-adjacent primary activation switches 82 before SCARA 24 and end effector coupler 22 will respond to operator commands. Depression of at least two primary activation switches 82 may prevent the accidental movement of SCARA 24 and end effector coupler 22 during a medical procedure.

Activated by primary button 78 and primary activation switch 82, load cell 64 may measure the force magnitude and/or direction exerted upon end effector coupler 22 by an operator, i.e. medical personnel. This information may be transferred to motors within SCARA 24 that may be used to move SCARA 24 and end effector coupler 22. Information as to the magnitude and direction of force measured by load cell 64 may cause the motors to move SCARA 24 and end effector coupler 22 in the same direction as sensed by load cell 64. This force-controlled movement may allow the operator to move SCARA 24 and end effector coupler 22 easily and without large amounts of exertion due to the motors moving SCARA 24 and end effector coupler 22 at the same time the operator is moving SCARA 24 and end effector coupler 22.

Secondary button 80, as illustrated in FIG. 6, may be disposed upon the end of activation assembly 60 closest to saddle joint 62. In some examples secondary button 80 may comprise a plurality of ridges. The plurality of ridges may be disposed adjacent to each other and may encircle connector 66. Additionally, secondary button 80 may be disposed upon secondary activation switch 84. Secondary activation switch 84, as illustrated in FIG. 7, may be disposed between secondary button 80 and connector 66. In some examples, secondary button 80 may be used by an operator as a “selection” device. During a medical operation, surgical robot 4 may notify medical personnel to certain conditions by display 34 and/or light indicator 28. Medical personnel may be prompted by surgical robot 4 to select a function, mode, and/or assess the condition of surgical system 2. Depressing secondary button 80 upon secondary activation switch 84 a single time may activate certain functions, modes, and/or acknowledge information communicated to medical personnel through display 34 and/or light indicator 28. Additionally, depressing secondary button 80 upon secondary activation switch 84 multiple times in rapid succession may activate additional functions, modes, and/or select information communicated to medical personnel through display 34 and/or light indicator 28. In some examples, at least two non-adjacent secondary activation switches 84 may be depressed before secondary button 80 may function properly. This requirement may prevent unintended use of secondary button 80 from accidental bumping by medical personnel upon activation assembly 60. Primary button 78 and secondary button 80 may use software architecture 86 to communicate commands of medical personnel to surgical system 2.

FIG. 8 illustrates a block diagram of components of a surgical system 800 configured according to some embodiments of the present disclosure, and which may correspond to the surgical system 2 above. Surgical system 800 includes platform subsystem 802, computer subsystem 820, motion control subsystem 840, and tracking subsystem 830. Platform subsystem 802 includes battery 806, power distribution module 804, connector panel 808, and charging station 810. Computer subsystem 820 includes computer 822, display 824, and speaker 826. Motion control subsystem 840 includes driver circuit 842, motors 850, 851, 852, 853, 854, stabilizers 855, 856, 857, 858, end effector connector 844, and controller 846. Tracking subsystem 830 includes position sensor 832 and camera converter 834. Surgical system 800 may also include a removable foot pedal 880 and removable tablet computer 890.

Input power is supplied to surgical system 800 via a power source which may be provided to power distribution module 804. Power distribution module 804 receives input power and is configured to generate different power supply voltages that are provided to other modules, components, and subsystems of surgical system 800. Power distribution module 804 may be configured to provide different voltage supplies to connector panel 808, which may be provided to other components such as computer 822, display 824, speaker 826, driver 842 to, for example, power motors 850-854 and end effector coupler 844, and provided to camera converter 834 and other components for surgical system 800. Power distribution module 804 may also be connected to battery 806, which serves as temporary power source in the event that power distribution module 804 does not receive power from an input power. At other times, power distribution module 804 may serve to charge battery 806.

Connector panel 808 may serve to connect different devices and components to surgical system 800 and/or associated components and modules. Connector panel 808 may contain one or more ports that receive lines or connections from different components. For example, connector panel 808 may have a ground terminal port that may ground surgical system 800 to other equipment, a port to connect foot pedal 880, a port to connect to tracking subsystem 830, which may include position sensor 832, camera converter 834, and marker tracking cameras 870. Connector panel 808 may also include other ports to allow USB, Ethernet, HDMI communications to other components, such as computer 822.

Control panel 816 may provide various buttons or indicators that control operation of surgical system 800 and/or provide information from surgical system 800 for observation by an operator. For example, control panel 816 may include buttons to power on or off surgical system 800, lift or lower vertical column 16, and lift or lower stabilizers 855-858 that may be designed to engage casters 12 to lock surgical system 800 from physically moving. Other buttons may stop surgical system 800 in the event of an emergency, which may remove all motor power and apply mechanical brakes to stop all motion from occurring. Control panel 816 may also have indicators notifying the operator of certain system conditions such as a line power indicator or status of charge for battery 806.

Computer 822 of computer subsystem 820 includes an operating system and software to operate assigned functions of surgical system 800. Computer 822 may receive and process information from other components (for example, tracking subsystem 830, platform subsystem 802, and/or motion control subsystem 840) in order to display information to the operator. Further, computer subsystem 820 may provide output through the speaker 826 for the operator. The speaker may be part of the surgical robot, part of a head-mounted display component, or within another component of the surgical system 2. The display 824 may correspond to the display 34 shown in FIGS. 1 and 2, or may be a head-mounted display which projects images onto a see-through display screen which forms an augmented reality image that is overlaid on real-world objects viewable through the see-through display screen.

Tracking subsystem 830 may include position sensor 832 and camera converter 834. Tracking subsystem 830 may correspond to the camera tracking system 6 of FIG. 3. The marker tracking cameras 870 operate with the position sensor 832 to determine the pose of DRAs 52. This tracking may be conducted in a manner consistent with the present disclosure including the use of infrared or visible light technology that tracks the location of active or passive elements of DRAs 52, such as LEDs or reflective markers, respectively. The location, orientation, and position of structures having these types of markers, such as DRAs 52, is provided to computer 822 and which may be shown to an operator on display 824. For example, as shown in FIGS. 4 and 5, a surgical saw 1240 having a DRA 52 or which is connected to an end effector coupler 22 having a DRA 52 tracked in this manner (which may be referred to as a navigational space) may be shown to an operator in relation to a three dimensional image of a patient's anatomical structure.

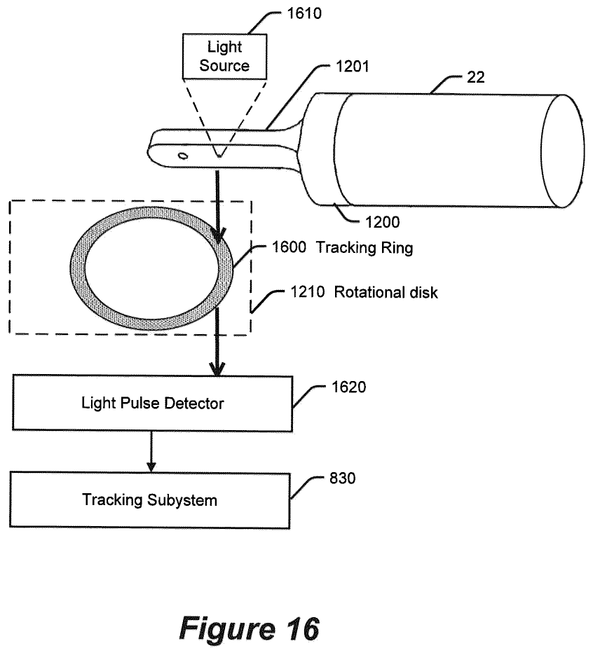

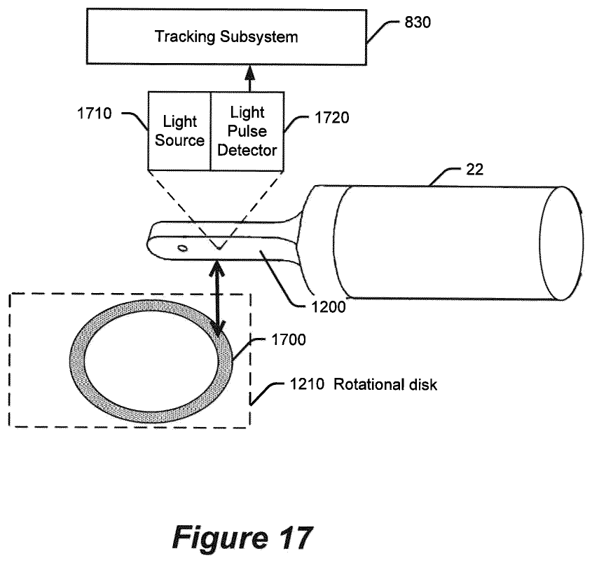

Alternatively or additionally, the tracking subsystem 830 tracks a pose, e.g., rotational motion, of a surgical saw connected to the passive end effector 1100 responsive to signaling from a light pulse detector and tracking ring that can be incorporated onto the passive end effector 1100, such as described below for FIGS. 16 and 17.

Motion control subsystem 840 may be configured to physically move vertical column 16, upper arm 18, lower arm 20, or rotate end effector coupler 22. The physical movement may be conducted through the use of one or more motors 850-854. For example, motor 850 may be configured to vertically lift or lower vertical column 16. Motor 851 may be configured to laterally move upper arm 18 around a point of engagement with vertical column 16 as shown in FIG. 2. Motor 852 may be configured to laterally move lower arm 20 around a point of engagement with upper arm 18 as shown in FIG. 2. Motors 853 and 854 may be configured to move end effector coupler 22 to provide translational movement and rotation along in about three-dimensional axes. The surgical planning computer 910 shown in FIG. 9 can provide control input to the controller 846 that guides movement of the end effector coupler 22 to position a passive end effector, which is connected thereto, with a planned pose (i.e., location and angular orientation relative to defined 3D orthogonal reference axes) relative to an anatomical structure that is to be cut during a surgical procedure. Motion control subsystem 840 may be configured to measure position of the passive end effector structure using integrated position sensors (e.g. encoders). In one of the embodiments, position sensors are directly connected to at least one joint of the passive end effector structure, but may also be positioned in another location in the structure and remotely measure the joint position by interconnection of a timing belt, a wire, or any other synchronous transmission interconnection.

FIG. 9 illustrates a block diagram of a surgical system computer platform 900 that includes a surgical planning computer 910 which may be separate from and operationally connected to a surgical robot 800 or at least partially incorporated therein according to some embodiments of the present disclosure. Alternatively, at least a portion of operations disclosed herein for the surgical planning computer 910 may be performed by components of the surgical robot 800 such as by the computer subsystem 820.

Referring to FIG. 9, the surgical planning computer 910 includes a display 912, at least one processor circuit 914 (also referred to as a processor for brevity), at least one memory circuit 916 (also referred to as a memory for brevity) containing computer readable program code 918, and at least one network interface 920 (also referred to as a network interface for brevity). The network interface 920 can be configured to connect to a C-Arm imaging device 104 in FIG. 10, an O-Arm imaging device 106 in FIG. 11, another medical imaging device, an image database 950 of medical images, components of the surgical robot 800, and/or other electronic equipment.

When the surgical planning computer 910 is at least partially integrated within the surgical robot 800, the display 912 may correspond to the display 34 of FIG. 2 and/or the tablet 890 of FIG. 8 and/or a head-mounted display, the network interface 920 may correspond to the platform network interface 812 of FIG. 8, and the processor 914 may correspond to the computer 822 of FIG. 8.

The processor 914 may include one or more data processing circuits, such as a general purpose and/or special purpose processor, e.g., microprocessor and/or digital signal processor. The processor 914 is configured to execute the computer readable program code 918 in the memory 916 to perform operations, which may include some or all of the operations described herein as being performed by a surgical planning computer.

The processor 914 can operate to display on the display device 912 an image of a bone that is received from one of the imaging devices 104 and 106 and/or from the image database 950 through the network interface 920. The processor 914 receives an operator's definition of where an anatomical structure, i.e. one or more bones, shown in one or more images is to be cut, such as by an operator touch selecting locations on the display 912 for planned surgical cuts or using a mouse-based cursor to define locations for planned surgical cuts.

The surgical planning computer 910 enables anatomy measurement, useful for knee surgery, like measurement of various angles determining center of hip, center of angles, natural landmarks (e.g. transepicondylar line, Whitesides line, posterior condylar line), etc. Some measurements can be automatic while some others be involve human input or assistance. This surgical planning computer 910 allows an operator to choose the correct implant for a patient, including choice of size and alignment. The surgical planning computer 910 enables automatic or semi-automatic (involving human input) segmentation (image processing) for CT images or other medical images. The surgical plan for a patient may be stored in a cloud-based server for retrieval by the surgical robot 800. During the surgery, the surgeon will choose which cut to make (e.g. posterior femur, proximal tibia etc.) using a computer screen (e.g. touchscreen) or augmented reality interaction via, e.g., a head-mounted display. The surgical robot 4 may automatically move the surgical saw to a planned position so that a target plane of planned cut is optimally placed within a workspace of the passive end effector interconnecting the surgical saw and the robot arm 20.

In some embodiments, the surgical system computer platform 900 can use two DRAs to tracking patient anatomy position: one on patient tibia and one on patient femur. The platform 900 may use standard navigated instruments for the registration and checks (e.g. a pointer similar to the one used in Globus ExcelsiusGPS system for spine surgery). Tracking markers allowing for detection of DRAs movement in reference to tracked anatomy can be used as well.

A particularly challenging task in knee surgery is how to plan the position of the implant in the knee and many surgeons struggle with this task on a computer screen which is a 2D representation of 3D anatomy. The platform 900 could address this problem by using a augmented reality (AR) head-mounted display to generate an implant overlay around the actual patient knee. For example, the surgeon can be operationally displayed a virtual handle to grab and move the implant to a desired pose and adjust planned implant placement. Afterward, during surgery, the platform 900 could render the navigation through the AR head-mounted display to show surgeon what is not directly visible. Also, the progress of bone removal, e.g., depth or cut, can be displayed in real-time. Other features that may be displayed through AR can include, without limitation, gap or ligament balance along a range of joint motion, contact line on the implant along the range of joint motion, ligament tension and/or laxity through color or other graphical renderings, etc.

The surgical planning computer 910, in some embodiments, can allow planning for use of standard implants, e.g., posterior stabilized implants and cruciate retaining implants, cemented and cementless implants, revision systems for surgeries related to, for example, total or partial knee and/or hip replacement and/or trauma.

The processor 912 may graphically illustrate on the display 912 one or more cutting planes intersecting the displayed anatomical structure at the locations selected by the operator for cutting the anatomical structure. The processor 912 also determines one or more sets of angular orientations and locations where the end effector coupler 22 should be positioned so a cutting plane of the surgical saw will be aligned with a target plane to perform the operator defined cuts, and stores the sets of angular orientations and locations as data in a surgical plan data structure. The processor 912 uses the known range of movement of the tool attachment mechanism of the passive end effector to determine where the end effector coupler 22 attached to the robot arm 20 needs to be positioned.

The computer subsystem 820 of the surgical robot 800 receives data from the surgical plan data structure and receives information from the camera tracking system 6 indicating a present pose of an anatomical structure that is to be cut and indicating a present pose of the passive end effector and/or surgical saw tracked through DRAs. The computer subsystem 820 determines a pose of the target plane based on the surgical plan defining where the anatomical structure is to be cut and based on the pose of the anatomical structure. The computer subsystem 820 generates steering information based on comparison of the pose of the target plane and the pose of the surgical saw. The steering information indicates where the passive end effector needs to be moved so the cutting plane of the saw blade becomes aligned with the target plane and the saw blade becomes positioned a distance from the anatomical structure to be cut that is within the range of movement of the tool attachment mechanism of the passive end effector.

As explained above, a surgical robot includes a robot base, a robot arm rotatably connected to the robot base, and at least one motor operatively connected to move the robot arm relative to the robot base. The surgical robot also includes at least one controller, e.g. the computer subsystem 820 and the motion control subsystem 840, connected to the at least one motor and configured to perform operations.

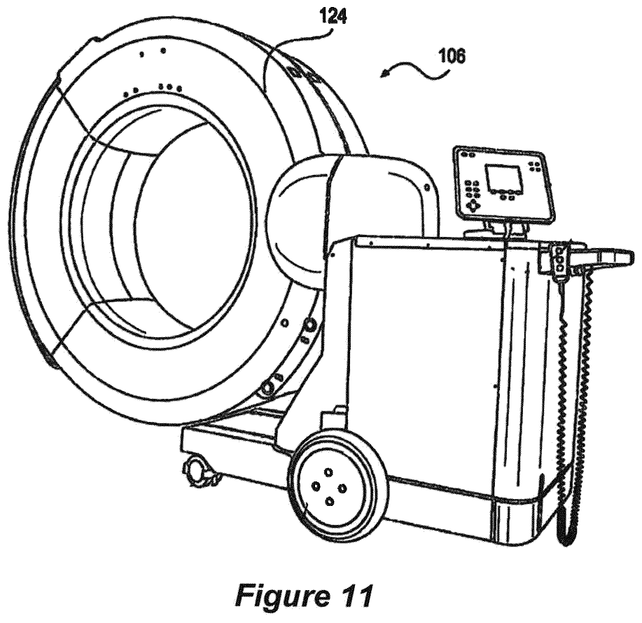

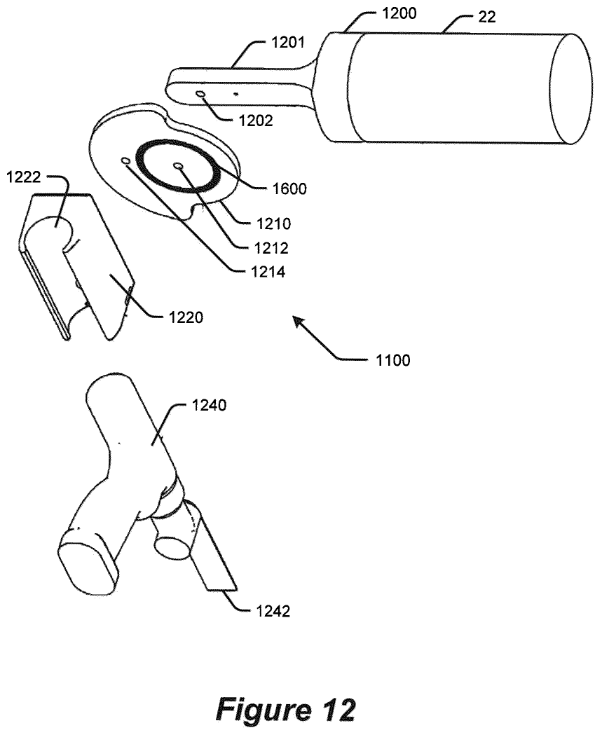

As will be explained in further detail below with regard to FIGS. 12-17, passive end effectors are disclosed that interconnect a surgical saw to the end effector coupler of the robot arm of a surgical robot. The passive end effectors include a rotational disk rotatably connected a base which is connected to the end effector coupler, and includes a saw attachment that us rotatably connected to the rotational disk. The saw attachment is configured to connect to a surgical saw having a saw blade configured to oscillate for cutting. The saw attachment rotates about the rotational disk and the rotational disk rotates about the base arm to constrain cutting of the saw blade to a range of movement along arcuate paths within a cutting plane.

In one embodiment, the controller(s) of the surgical robot controls movement of the at least one motor based on the steering information to reposition the passive end effector so the cutting plane of the saw blade becomes aligned with the target plane and the saw blade becomes positioned a distance from the anatomical structure to be cut that is within the range of movement of the saw blade provided by the rotational disk.

In another embodiment, the controller(s) of the surgical robot provide the steering information to a display device for display to guide operator movement of the passive end effector so the cutting plane of the saw blade becomes aligned with the target plane and so the saw blade becomes positioned the distance from the anatomical structure, which is to be cut, that is within the range of movement of the passive end effector. The display device may correspond to the display 824 (FIG. 8), the display 34 of FIG. 1, and/or a head-mounted display.

For example, the steering information may be displayed on a head-mounted display which projects images onto a see-through display screen which forms an augmented reality image that is overlaid on real-world objects viewable through the see-through display screen. The controller(s) of the surgical robot may display a graphical representation of the target plane with a pose overlaid on a bone and with a relative orientation there between corresponding to the surgical plan for how the bone is planned to be cut. Alternatively or additionally, a graphical representation of the cutting plane of the saw blade can be displayed so that an operator may more easily align the cutting plane with the planned target plane for cutting the bone. The operator may thereby visually observe and perform movements to align the cutting plane of the saw blade with the target plane so the saw blade becomes positioned at the planned pose relative to the bone and within a range of movement of the tool attachment mechanism of the passive end effector.

An automated imaging system can be used in conjunction with the surgical planning computer 910 and/or the surgical system 2 to acquire pre-operative, intra-operative, post-operative, and/or real-time image data of a patient. Example automated imaging systems are illustrated in FIGS. 10 and 11. In some embodiments, the automated imaging system is a C-arm 104 (FIG. 10) imaging device or an O-arm® 106 (FIG. 11). (O-arm® is copyrighted by Medtronic Navigation, Inc. having a place of business in Louisville, Colo., USA) It may be desirable to take x-rays of a patient from a number of different positions, without the need for frequent manual repositioning of the patient which may be required in an x-ray system. C-arm 104 x-ray diagnostic equipment may solve the problems of frequent manual repositioning and may be well known in the medical art of surgical and other interventional procedures. As illustrated in FIG. 10, a C-arm includes an elongated C-shaped member terminating in opposing distal ends 112 of the “C” shape. C-shaped member is attached to an x-ray source 114 and an image receptor 116. The space within C-arm 104 of the arm provides room for the physician to attend to the patient substantially free of interference from the x-ray support structure.

The C-arm is mounted to enable rotational movement of the arm in two degrees of freedom, (i.e. about two perpendicular axes in a spherical motion). C-arm is slidably mounted to an x-ray support structure, which allows orbiting rotational movement of the C-arm about its center of curvature, which may permit selective orientation of x-ray source 114 and image receptor 116 vertically and/or horizontally. The C-arm may also be laterally rotatable, (i.e. in a perpendicular direction relative to the orbiting direction to enable selectively adjustable positioning of x-ray source 114 and image receptor 116 relative to both the width and length of the patient). Spherically rotational aspects of the C-arm apparatus allow physicians to take x-rays of the patient at an optimal angle as determined with respect to the particular anatomical condition being imaged.

The O-arm® 106 illustrated in FIG. 11 includes a gantry housing 124 which may enclose an image capturing portion, not illustrated. The image capturing portion includes an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of the patient to be acquired from multiple directions or in multiple planes.

The O-arm® 106 with the gantry housing 124 has a central opening for positioning around an object to be imaged, a source of radiation that is rotatable around the interior of gantry housing 124, which may be adapted to project radiation from a plurality of different projection angles. A detector system is adapted to detect the radiation at each projection angle to acquire object images from multiple projection planes in a quasi-simultaneous manner. The gantry may be attached to a support structure O-arm® support structure, such as a wheeled mobile cart with wheels, in a cantilevered fashion. A positioning unit translates and/or tilts the gantry to a planned position and orientation, preferably under control of a computerized motion control system. The gantry may include a source and detector disposed opposite one another on the gantry. The source and detector may be secured to a motorized rotor, which may rotate the source and detector around the interior of the gantry in coordination with one another. The source may be pulsed at multiple positions and orientations over a partial and/or full three hundred and sixty degree rotation for multi-planar imaging of a targeted object located inside the gantry. The gantry may further comprise a rail and bearing system for guiding the rotor as it rotates, which may carry the source and detector. Both and/or either O-arm® 106 and C-arm 104 may be used as automated imaging system to scan a patient and send information to the surgical system 2.

Images captured by the automated imaging system can be displayed a display device of the surgical planning computer 910, the surgical robot 800, and/or another component of the surgical system 2.

Various embodiments of passive end effectors that are configured for use with a surgical system are now described in the context of FIGS. 12-17.

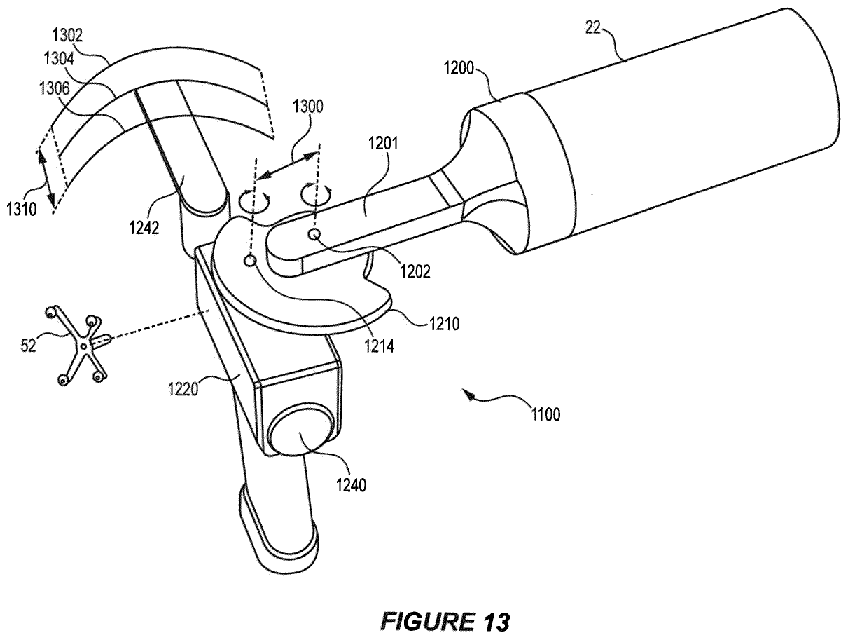

FIGS. 12 illustrates an exploded view of components of a passive end effector 1100 that can be connected to a surgical saw 1240 and which are configured in accordance with some embodiments of the present disclosure. FIG. 13 illustrates the assembled passive end effector of FIG. 12 connected to the surgical saw 1240 and configured in accordance with some embodiments of the present disclosure.

Referring to FIGS. 12 and 13, the passive end effector 1100 includes a base 1200, a rotational disk 1210, and a saw attachment 1220. The base 1200 is configured to attach to an end effector coupler 22 of the robot arm that is positioned by a surgical robot. Various attachment mechanisms may be used to firmly attach the base 1200 to the end effector coupler 22, removing backlash and ensuring suitable stiffness. Clamping mechanisms which may be used to attach the base 1200 to the end effector coupler 22 can include but are not limited to toggle joint mechanisms or locking screw(s). The base 1200 includes a base arm 1201 that extends away from the end effector coupler 22. The rotational disk 1210 is rotatably connected to the base arm and rotates about a first location 1212 on the rotational disk relative to a location 1202 on the base arm 1201. The rotational disk 1210 may be connected to the base arm 1201 by, for example, a bolt extending through locations 1212 and 1202 of the rotational disk 1210 and base arm 1201, respectively. The rotational disk 1210 may alternatively or additionally connect to the base arm 1201 through various mechanisms that can include, but are not limited to, a screw, clamp, latch, tie, or press fit.

The saw attachment 1220 is rotatably connected to the rotational disk 1210 and rotates about a second location 1214 on the rotational disk 1210. The first location 1212 on the rotational disk 1210 is spaced apart from the second location 1214 on the rotational disk 1210. The saw attachment 1220 is configured to connect to the surgical saw 1240 having a saw blade 1242 configured to oscillate for cutting. An example embodiment of the saw attachment 1222 is illustrated in FIG. 12 as having a cylindrical-shaped channel 1222 adapted to receive and retain a corresponding cylindrical-shaped portion of a housing of the surgical saw 1240. The saw attachment 1222 may alternatively or additionally connect to the surgical saw 1240 through various mechanisms that can include, but are not limited to, a screw, nut and bolt, clamp, latch, tie, press fit, or magnet. The saw attachment 1240 rotates about the rotational disk 1210 and the rotational disk 1210 rotates about the base arm 1201 to constrain cutting of the saw blade 1242 to a range of movement along arcuate paths within a cutting plane.

For example, FIG. 13 illustrates three sequentially generated arcuate paths made by a surgeon with the end of the saw blade 1242 by moving the surgical saw 1240 while connected to the saw attachment 1220. The surgical saw 1240 is rotated relative to rotational disk 1210 about axis 1214 to make an arcuate cut along path 1306 by the end of the saw blade 1242 into an anatomical structure. The surgical saw 1240 is then thrust forward toward the anatomical structure through rotation of the rotational disk 1210 relative to the base arm 1201 about axis 1202. The surgical saw 1240 is again rotated relative to rotational disk 1210 about axis 1214 to provide another arcuate cut by the end of the saw blade 1242 deeper into the anatomical structure along path 1304. The surgical saw 1240 is then thrust further forward toward the anatomical structure through rotation of the rotational disk 1210 relative to the base arm 1201 about axis 1202. The surgical saw 1240 is again rotated relative to rotational disk 1210 about axis 1214 to provide another arcuate cut deeper into the anatomical structure along path 1302. A surgeon can more continuously thrust and rotate the surgical saw 1240 to perform cutting of the anatomical structure with arcuate movements of the saw blade.

The distance 1300 between the location 1214, where the saw attachment 1220 connects to the rotational disk 1210, and the location 1202, where the rotational disk 1210 connects to the base arm 1201, constrains the range of thrusting motion of the end of the saw blade 1242 and, correspondingly, controls the depth of cut that can be made by the surgical saw 1240 into an anatomical structure while the surgical robot maintains, e.g., locks, the robot arm 20 (FIG. 2) and end effector coupler 22 with a fixed pose relative to the anatomical structure. For example, as shown in FIG. 13, the depth of cut 1310 made by the end of the saw blade 1242 is constrained by the extent of rotational movement of the rotational disk 1210 relative to the base arm 1200 that occurs when thrusting toward the anatomical structure. At least some embodiments, the depth of cut is constrained to be not greater than the distance 1300 between the connection locations 1214 and 1202.

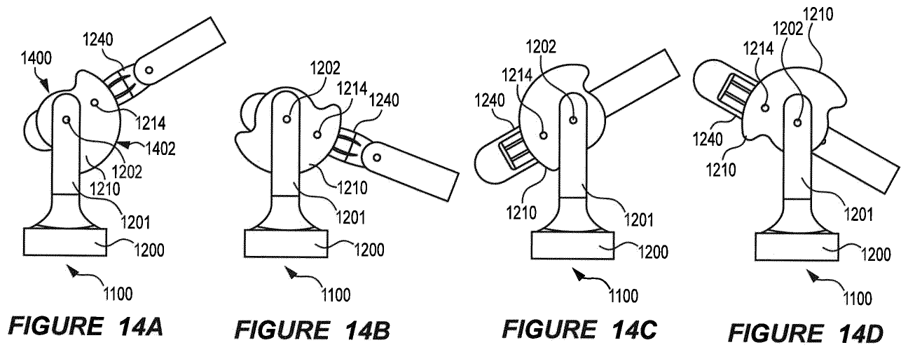

FIGS. 14a-14d illustrate a sequence of top views of the passive end effector 1100 and surgical saw 1240 of FIG. 13 in which the surgical saw 1240 is rotated about the rotational disk 1210 and the rotational disk 1210 is sequentially rotated clockwise rotated about the base arm 1201 to provide a range of movement of the saw blade along arcuate paths within a cutting plane in accordance with some embodiments of the present disclosure. The sequence of top views illustrates four different orientations of the location 1214, where the saw attachment 1220 fixed to the surgical saw 1240 connects to the rotational disk 1210, relative to the location 1210, where the rotational disk 1210 connects to the base arm 1201. The distance that the tip of the saw 1240 can be thrust to the right of the base arm 1201 decreases from when the location 1214 is to the right of location 1202, as shown in FIGS. 14a and 14b, relative to when the location 1214 is to the left of location 1202, as shown in FIGS. 14c and 14d.

During a non-limiting example surgical procedure, a surgeon may repetitively rotate the saw blade back and forth while slowly rotating the rotational disk 1210 clockwise from the orientation illustrated in FIG. 14c to the orientation illustrated in FIG. 14d, which thrusts the end of the surgical saw along an arcuate cutting path into an anatomical structure located to the right of the base arm 1201. The surgeon may then cut deeper into the anatomical structure by continuing to rotate the rotational disk 1210 in a clockwise direction from the orientation illustrated in FIG. 14a to the orientation illustrated in FIG. 14b while repetitively rotating the saw blade back and forth, which further thrusts the end of the surgical saw along an arcuate cutting path into the anatomical structure.

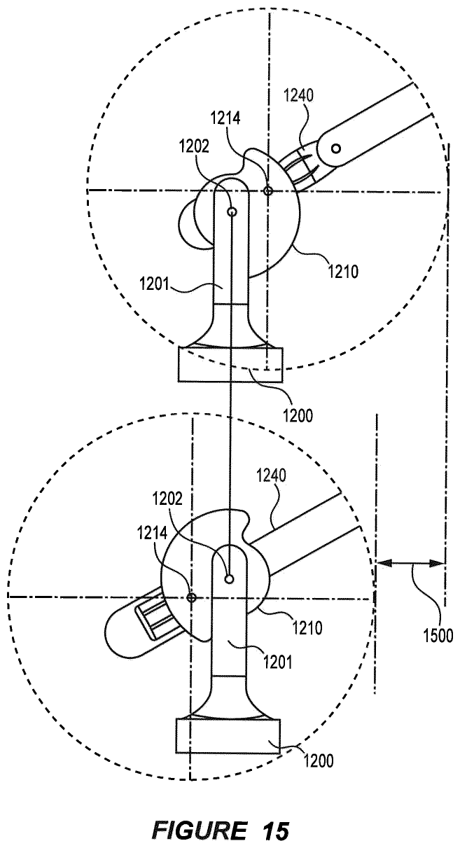

FIG. 15 illustrates a combination of the top views of the passive end effector and surgical saw of FIGS. 14a and 14c to show ranges of the movement of the saw blade along a horizontal axis that are provided by rotation of the surgical saw 1240 about the rotational disk 1210 and rotation of the rotational disk 1210 about the base arm 1201. In the top view configuration it is observed that while the location 1214 is maintained at a fixed angle to the right of location 1202, the tip of the saw blade can be rotated along cutting circle 1502. In the bottom view configuration the rotational disk 1210 has been rotated 180° with the location 1214 now maintained at another fixed angle to the left of location 1202, which results in the tip of the saw blade being rotatable along another cutting circle 1504. The cutting circle 1502 of the top view configuration extends further to the right than the cutting circle 1504 of the bottom view configuration, which corresponds to a difference in cutting depth 1500 between the top and bottom view configurations. As explained above, the distance between the locations 1202 and 1214 controls the cutting depth 1500. Increasing the distance between locations 1202 and 1214 increases the cutting depth 1500, and in contrast decreasing the distance between locations 1202 and 1214 decreases the cutting depth 1500.

In some embodiments, the distance that the location 1202 on the rotational disk 1210, where the rotational disk 1210 connects to the base arm 1201, is spaced apart from the location 1214 on the rotational disk 1210, where the saw attachment 1220 connects to the rotational disk 1210, is less than the first radius. For many orthopedic surgeries, it has been determined that a distance that the location 1202 on the rotational disk 1210 is spaced apart from the location 1214 on the rotational disk 1202 should preferably be within a range of at least 1 inch to not greater than 2.5 inches so the saw blade can be moved from a starting location where the surgical robot helped position the passive end effector 1100 relative to an anatomical structure to be cut, through a range of thrusting distances that constrain the depth of cutting of the anatomical structure to avoid excessive cutting beyond the anatomical structure, e.g., femur. It has further been determined that for some types of orthopedic surgeries, such as knee surgeries, the distance that the location 1202 on the rotational disk 1210 is spaced apart from the location 1214 on the rotational disk 1210 should preferably be within a range of at least 1.5 inch to not greater than 2 inches.

In some embodiments, the rotational disk has a recessed sector portion that facilitates maintaining a desired minimum distance from the anatomical structure being cut while the rotational disk is being initially positioned and then rotated during surgery. In the example shown in FIG. 14a, the rotational disk 1210 includes a first sector portion and a second sector portion, where the first sector portion has a first radius between the location 1202 and an edge surface 1400 of the first section portion, and where the second sector portion has a second radius between the location 1202 and an edge surface 1402 of the second sector portion. The second radius is larger than the first radius. The saw attachment is rotatably connected to the second portion of the rotational disk with the location 1214, where the saw attachment 1220 connects to the rotational disk 10, being closer to the edge surface 1402 of the second portion than to the edge surface 1400 of the first portion.

During surgery, the surgical robot 4 can be configured to move the end effector coupler 22, and the passive end effector and attached surgical saw, automatically to a position close to a knee or other anatomical structure, so that all bone to be cut is within the workspace of the passive end effector. This position depends on the cut to be made and the surgery planning and implant construction.

When the surgical robot 4 achieves a planned position, it holds the position (either on brakes or active motor control) and does not move during the particular bone cut. It is the passive end effector that allows movement of the saw blade of the surgical saw along the planned target plane. Such planar cuts are particularly useful for classical total knee arthroplasty where all bone cuts are planar. In partial knee arthroplasty there are special types of implants, called “on-lay” which can be in conjunction with saw-prepared bone surfaces. The various passive end effectors have mechanical structure that can ensure precision of guidance during cuts, with higher precision than classical jigs, and provide sufficient range of workspace range to cut all the bone that is planned and while provide sufficient transverse stiffness (corresponding to locked DOF) despite possibly significant amount of vibrations originating from the surgical saw in addition to forces applied by the surgeon and bone reactionary forces.

It is preferable to measure the passive end effector position because it enables the surgical robot 4 to inform the surgeon how much bone has been removed (procedure advancement). One way to provide real-time information on bone removal is for the surgical robot 4 to measure where the saw blade passed in reference to the bone because the blade can pass only where the bone has been cut.

In one embodiment, a conventional sagittal saw mechanism can be used with the surgical system computer platform 900 with little or no changes. The potential changes would involve adapting an external shield to enable easy attachment of the surgical saw to the passive end effector but would not necessarily involve changes in the internal mechanics. The passive end effector may be configured to connect to a conventional sagittal saw provided by, for example, DeSoutter company.

To prevent the saw from unintentional passive end effector movement when the surgical robot 4 positions the passive end effector, e.g., to prevent the surgical saw from falling on the patient due to gravitational forces, the passive end effector can include a lock mechanism that moves between engaged and disengaged operations. While engaged, the lock mechanism prevents movement of the saw blade with respect to the robot end effector coupler, either directly by locking the degree of freedoms (DOFs) of the surgical saw, or indirectly by braking or locking specifics joints of the passive end effector. While disengaged, the first and second planar mechanisms of the passive end effector can be moved relative to the base without interference from the lock mechanism. The lock mechanism may also be used when a surgeon holds the surgical saw and controls the surgical robot 4 movement by applying forces and torques to the surgical saw. The surgical robot 4, using the load cell 64 of FIGS. 6 and 7 integrated in the distal end of the robot arm 22, measures forces and torques that are applied and generates responsive forces and torques on the robot arm 22 so the surgeon can more easily move the passive end effector back and forth, left and right, apply rotations around various axes.

As explained above, a surgical system (e.g., surgical system 2 in FIGS. 1 and 2) includes the surgical robot (e.g., surgical robot 4 in FIGS. 1 and 2) and the tracking system (e.g., camera tracking system 6 in FIGS. 1 and 3).

The tracking system can be configured to determine a pose of an anatomical structure that is to be cut and to determine a pose of a saw blade of a surgical saw connected to a passive end effector supported by the robot arm. The tracking system may determine the range of movement of the saw blade along arcuate paths within the cutting plane while connected to the passive end effector.

The surgical robot includes the robot base and the robot arm that is rotatably connected to the robot base and configured to position the passive end effector. At least one motor is operatively connected to move the robot arm relative to the robot base. At least one controller is connected to control movement of the at least one motor. The controller(s) of the surgical robot is configured to determine a pose of a target plane based on a surgical plan defining where the anatomical structure is to be cut and based on the pose of the anatomical structure. The controller(s) is further configured to generate steering information based on comparison of the pose of the target plane and the determined range of movement of the saw blade along arcuate paths within the cutting plane. The steering information indicates where the passive end effector needs to be moved to position the cutting plane of the saw blade to be aligned with the target plane and so the saw blade is within the range of movement from the anatomical structure to be cut.

In some further embodiments, the controller(s) of the surgical robot controls movement of the motor(s) based on the steering information to reposition the passive end effector so the cutting plane of the saw blade becomes aligned with the target plane and the saw blade becomes positioned a distance from the anatomical structure to be cut that is within the range of movement of the saw blade provided by the rotational disk.

In some alternative or further embodiments, the controller(s) of the surgical robot provides the steering information to a display device for display to guide operator movement of the passive end effector so the cutting plane of the saw blade becomes aligned with the target plane and so the saw blade becomes positioned a distance from the anatomical structure to be cut that is within the range of movement of the saw blade provided by rotation of the rotational disk.

As explained above, some surgical systems can include head-mounted display devices that can be worn by a surgeon, nurse practitioner, and/or other persons assisting with the surgical procedure. A surgical system can display information that allows the wearer to position the passive end effector more accurately and/or to confirm that it has been positioned accurately with the saw blade aligned with the target plane for cutting a planned location on an anatomical structure. The operation to provide the steering information to the display device, may include generating the steering information for display on a head-mounted display device having a see-through display screen which displays the steering information as an overlay on the anatomical structure to be cut to guide operator movement of the passive end effector so the cutting plane of the saw blade becomes aligned with the target plane and the saw blade becomes positioned the distance from the anatomical structure within the range of movement of the saw blade provided by rotation of the rotational disk.

The operation to generate the steering information for display on the head-mounted display device, may include generating a graphical representation of the target plane that is displayed as an overlay anchored to and aligned with the anatomical structure that is to be cut, and generating another graphical representation of the cutting plane of the saw blade that is displayed as an overlay anchored to and aligned with the saw blade. A wearer may thereby move the surgical saw to provide visually observed alignment between the graphically rendered target plane and the graphically rendered cutting plane.

The operation to generate the steering information for display on the head-mounted display device, may include generating a graphical representation a depth of cut made by the saw blade into the anatomical structure being cut. Thus, the wearer can use the graphical representation of depth of cut to better monitor how the saw blade is cutting through bone despite direct observation of the cutting being obstructed by tissue or other structure.

The tracking system can be configured to determine the pose of the anatomical structure that is to be cut by the saw blade based on determining a pose of tracking markers, e.g., DRAs, that are attached to the anatomical structure, and can be configured to determine a pose of the surgical saw based on determining a pose of tracking markers connected to at least one of the surgical saw and the passive end effector. The tracking system can be configured to determine the pose of the surgical saw based on rotary position sensors which are configured to measure rotational positions of the first and second planar mechanisms during movement of the tool attachment mechanism within the working plane. As explained above, position sensors may be directly connected to at least one rotational connection, e.g., location 1202 and/or location 1214) of the passive end effector structure, but may also be positioned in another location in the structure and remotely measure the joint position by interconnection of a measurement belt, a wire, or any other synchronous transmission interconnection.

Another technical approach that can be used to facilitate tracking of the pose of surgical saw is to utilize a light source that shines light onto a tracking ring which is on the rotational disk 1210, and a light pulse detector that detects pulses of light that can be passed through the tracking ring or reflected therefrom. The tracking ring can be configured to generate pulses of light as the rotational disk is rotated relative to the light source. The tracking system can be configured to determine the pose of the anatomical structure to be cut by the saw blade based on a determination of a pose of tracking markers that are attached to the anatomical structure, and configured to determine the range of movement of the saw blade along arcuate paths within the cutting plane while connected to the saw attachment based on counting pulses of light indicated by signaling received from the light pulse detector.

FIG. 16 illustrates a light source 1610, a tracking ring 1600, and a light pulse detector 1620 that are configured in accordance with one embodiment to provide input to the tracking system 830 for determining an arcuate path through which the saw blade moves. The tracking ring 1600 is also shown in FIG. 12. Referring to FIGS. 12 and 16, the light source 1610 can be connected to the base arm 1201 and oriented to emit light toward the tracking ring 1600. The tracking ring 1600 is within the rotational disk 1210 and extends from a first side of the rotational disk 1210, which is adjacent to the base arm 1201, to a second side of the rotational disk 1210, which is adjacent to the saw attachment 1220. The tracking ring 1600 includes circumferentially spaced apart alternating areas of light translucent material and light opaque material. The light translucent material allows light from the light source 1610 to pass through from the first side to the second side. In contrast, the light opaque material at least substantially prevents light from the light source 1610 from passing through from the first side to the second side.

The light pulse detector 1620 is aligned to detect light pulses formed as light alternatively passes through the light translucent material of the tracking ring 1600 and as light is at least substantially prevented from passing through the light opaque material of the tracking ring 1600 while the rotational disk 1210 is rotated relative to the base arm 1201. The light pulse detector 1620 may be connected to the rotational disk 1210, the saw attachment 1220, or another structure of the surgical robot. The tracking system 830 is connected to receive signaling from the light pulse detector 1620, and is configured to determine an arcuate path within the cutting plane through which the saw blade moves based on the signaling from the light pulse detector 1620.

FIG. 17 illustrates a light source 1710, a tracking ring 1700, and a light pulse detector 1720 that are configured in accordance with another embodiment to provide input to the tracking system 830 for determining an arcuate path through which the saw blade moves. Referring to FIG. 17, the light source 1710 can be connected to the base arm 1201 and oriented to emit light toward the tracking ring 1700. The tracking ring 1700 is on a side of the rotational disk 1210 adjacent to the base arm 1201, and includes circumferentially spaced apart alternating areas of a first material that reflects incident light from the light source 1710 in a defined direction and a second material that at least substantially inhibits reflection of the incident light from the light source 1710 in the defined direction. The light pulse detector 1720 can be connected to the base arm 1201 and aligned to detect light pulses reflected in the first direction from the tracking ring 1700 while the rotational disk 1210 is rotated relative to the base arm 1201. The tracking system 830 is connected to receive signaling from the light pulse detector 1720, and is configured to determine an arcuate path within the cutting plane through which the saw blade moves based on the signaling from the light pulse detector 1720.

In some other embodiments, the tracking system 830 is configured to determine the pose of the saw blade based on rotary position sensors connected to measure rotation of the rotational disk relative to the base arm and/or to measure rotation of the saw attachment relative to the rotational disk. Example types of rotary position sensors that can be used with passive end effectors herein can include, but are not limited to: potentiometer sensor; capacitive encoder; rotary variable differential transformer (RVDT) sensor; linear variable differential transformer (LVDT) sensor; Hall effect sensor; and encoder sensor.

### Further Definitions and Embodiments

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

What is claimed is:

1. A robotic surgical system comprising:
a robot arm;
an end effector configured to be attached to the robot arm and having:
a base adapted to be moved by the robot arm of a surgical robot;
a base arm extending from the base;
a tracking system;
a surgical cutter for cutting a bone with a cutting element along a plane defined by rotation of the cutting element about the cutter axis, the surgical cutter having a cutter dynamic reference array (DRA) whose pose is trackable by the tracking system;
a patient DRA attachable to the patient and trackable by the tracking system such that the tracking system determines a pose of the surgical cutter relative to the patient based on tracking the cutter DRA and the patient DRA.

2. The robotic surgical system of claim 1, wherein the end effector includes an end effector DRB for tracking by the tracking system.

3. The robotic surgical system of claim 1, wherein the end effector includes a sensor configured to sense a rotation of the surgical cutter and determine a rotational position of the surgical cutter relative to the end effector.

4. The robotic surgical system of claim 1, further comprising a controller configured to:
determine a pose of a target plane based on a surgical plan defining where an anatomical structure is to be cut and based on the patient DRB, and
generate steering information based on comparison of the pose of the target plane, the steering information indicating where the end effector needs to be moved to position the cutting plane of the cutting element to be aligned with the target plane.

5. The surgical system of claim 4, wherein the controller is further configured to control movement of the robot arm based on the steering information to reposition the end effector so that the cutting plane of the cutting element becomes aligned with the target plane and the cutting element becomes positioned a selected distance from the anatomical structure to be cut that is within the range of movement of the cutting element.

6. The surgical system of claim 3, wherein the sensor includes a light source connected to the base arm and configured to emit light toward the surgical cutter.

7. The surgical system of claim 6, further comprising a rotor rotatably coupled between the base arm and the surgical cutter, wherein the rotor includes a tracking ring having circumferentially spaced apart alternating areas of light translucent material and light opaque material;
the system further comprising a light pulse detector aligned to detect light pulses that have been reflected from the tracking ring.

8. The surgical system of claim 7, wherein the tracking system is configured to determine a path within the cutting plane through which the cutting element moves based on the detected light pulses by the light pulse detector.

9. A robotic surgical system comprising:
a robot base;
a plurality of robot arms;
an end effector configured to be attached to a last one of the robot arms and having:
a base adapted to be moved by the last robot arm of a surgical robot;
a base arm extending from the base;
a tracking system having a pair of optical cameras;
a surgical cutter for cutting a bone with a cutting element along a plane defined by rotation of the cutting element about the cutter axis, the surgical cutter having a cutter dynamic reference array (DRA) whose pose is trackable by the tracking system, the cutter DRB containing optical markers for tracking by the cameras;
a patient DRA attachable to the patient and trackable by the tracking system such that the tracking system determines a pose of the surgical cutter relative to the patient based on tracking the cutter DRA and the patient DRA, the patient DRB containing optical markers for tracking by the cameras.

10. The robotic surgical system of claim 9, wherein the end effector includes an end effector DRB for tracking by the tracking system.

11. The robotic surgical system of claim 9, wherein the end effector includes a sensor configured to sense a rotation of the surgical cutter and determine a rotational position of the surgical cutter relative to the end effector.

12. The robotic surgical system of claim 9, further comprising a controller configured to:
determine a pose of a target plane based on a surgical plan defining where an anatomical structure is to be cut and based on the patient DRB, and
generate steering information based on comparison of the pose of the target plane, the steering information indicating where the end effector needs to be moved to position the cutting plane of the cutting element to be aligned with the target plane.

13. The surgical system of claim 12, wherein the controller is further configured to control movement of the robot arms based on the steering information to reposition the end effector so that the cutting plane of the cutting element becomes aligned with the target plane and the cutting element becomes positioned a selected distance from the anatomical structure to be cut that is within the range of movement of the cutting element.

14. The surgical system of claim 11, wherein the sensor includes a light source connected to the base arm and configured to emit light toward the surgical cutter.

15. The surgical system of claim 14, further comprising a rotor rotatably coupled between the base arm and the surgical cutter, wherein the rotor includes a tracking ring having circumferentially spaced apart alternating areas of light translucent material and light opaque material;
the system further comprising a light pulse detector aligned to detect light pulses that have been reflected from the tracking ring.

16. The surgical system of claim 15, wherein the tracking system is configured to determine a path within the cutting plane through which the cutting element moves based on the detected light pulses by the light pulse detector.
